(19) United States
(12) Patent Application Publication
(10) Pub. No.: US 2009/0131720 A1
(43) Pub. Date: May 21, 2009

(54) PROCESS FOR THE SYNTHESIS OF HYDROXYL ARYLAMINES

(75) Inventor: Jennifer A. COGGAN, Cambridge (CA)

Correspondence Address: OLIFF & BERRIDGE, PLC., P.O. BOX 320850, ALEXANDRIA, VA, 22320-4850, US

(73) Assignee: XEROX CORPORATION, Norwalk, CT (US)

(21) Appl. No.: 11/940,718

(22) Filed: Nov. 15, 2007

(51) Int. Cl. C07C 211/33

(52) U.S. Cl. 564/305

(57) ABSTRACT

A process for forming a hydroxyl triarylamine compound includes reacting a halogenated aryl aldehyde with an aldehyde protecting agent to form a halogenated protected aryl aldehyde compound, and reacting the halogenated protected aryl aldehyde compound with an amine in the presence of a suitable catalyst, then reducing the resulting aldehyde triarylamine to form the hydroxyl triarylamine.

### RELATED APPLICATIONS

Commonly assigned, U.S. patent application Ser. No. 11/734,593 filed Apr. 12, 2007, describes a process for forming a triarylamine compound comprising reacting a halogenated aryl alcohol with an alcohol protecting agent and a first base to form a halogenated protected aryl alcohol compound, and reacting the halogenated protected aryl alcohol compound with an amine in the presence of a suitable catalyst and a second base.

Commonly assigned, U.S. patent application Ser. No. 11/563,931 filed Nov. 28, 2006, describes a process for forming a triarylamine compound, comprising reacting an aniline and an arylchloride in the presence of a palladium ligated catalyst and a base.

Commonly assigned, U.S. patent application Ser. No. 11/563,873 filed Nov. 28, 2006, describes a process for forming a diarylamine compound, comprising reacting an aniline and an arylbromide in the presence of a palladium ligated catalyst and a base.

Commonly assigned, U.S. patent application Ser. No. 11/263,671 filed Nov. 1, 2005, describes a process for the preparation of a tertiary arylamine compound, comprising reacting an arylhalide and an arylamine in an ionic liquid in the presence of a catalyst.

Commonly assigned, U.S. patent application Ser. No. 10/992,690 filed Nov. 22, 2004, describes a process for forming a tertiary arylamine compound, comprising reacting an arylbromide and an arylamine.

The appropriate components and process aspects of each of the foregoing, such as the arylamine precursor materials, may be selected for the present disclosure in embodiments thereof. The entire disclosures of the above-mentioned applications are totally incorporated herein by reference.

### TECHNICAL FIELD

This disclosure is generally directed to improved chemical processes for the synthesis of arylamine compounds such as hydroxyl triarylamine compounds. In particular, this disclosure provides a three-step method for producing a hydroxyl triarylamine molecule directly by the reaction of an mono- or di-arylamine, such as an aniline, with a halogenated aryl aldehyde, using a halogenated protected aryl aldehyde compound as an intermediate, and then reducing the aryl aldehyde to form the hydroxyl triarylamine compound.

### BACKGROUND

Image-forming devices such as copiers, printers and facsimiles include known electrophotographic systems in which charging, exposure, development, transfer, etc., are carried out using electrophotographic photoreceptors.

Such photoreceptors are known to include several layers, generally including: a substrate, an undercoating layer, an intermediate layer, an optional charge blocking layer, a charge generating layer over an undercoating layer and/or a blocking layer, a charge transport layer and an optional protective overcoat layer. These layers can be in a variety of orders to make up a functional device, and sometimes multiple layers can be combined in a single or mixed layer.

In the charge transport layer and the optional protective overcoat layer, hole transport molecules may be dispersed in a polymer binder. The hole transport molecules provide hole or electron transport properties, while the electrically inactive polymer binder provides mechanical properties.

Imaging members are generally exposed to repetitive electrophotographic cycling, which subjects the exposed charge transport layer or protective overcoat layer thereof to mechanical abrasion, chemical attack and heat. This repetitive cycling leads to gradual deterioration in the mechanical and electrical characteristics of the exposed charge transport layer.

In light of this deterioration, one type of protective overcoat layer has been used to provide increased crack, abrasion and scratch resistance of the photoreceptor when the photoreceptor is in a belt configuration. The layer is generally made up of three main components: a polyol binder, a melamine-formaldehyde curing agent and a hole transport material such as dihydroxymethylated triphenylamine.

The production of a number of arylamine compounds, such as dihydroxymethylated triphenylamine that is useful as the hole transport material in electrophotographic photoreceptors, often involves synthesis of intermediate materials that are generally costly and/or time-consuming to produce, and some of which involve a multi-step process.

One such class of arylamine compounds are triarylamines. Certain triarylamine compounds may be produced by reaction of an aniline with an aryliodide under traditional Ullman conditions (copper catalyst, high temperature, long reaction time) or the so-called ligand-accelerated Ullman reaction that uses lower reaction temperatures but is still limited to the use of aryliodides (see for example U.S. Pat. Nos. 5,902,901; 5,723,671; 5,723,669; 5,705,697; 5,654,482; and 5,648,542). Aryliodides tend to be very expensive reagents. Furthermore, both of these reactions usually require lengthy and costly purification processes.

Furthermore, typical reaction schemes for producing substituted triarylamines utilize the Vilsmeier reaction. The Vilsmeier reaction unfortunately includes reagents such as POCl₃ or POBr₃ that are very corrosive, and/or use hydrogen reduction reactions that can be very dangerous. These drawbacks, while nominal in a laboratory scale, pose significant challenges in scaling up a reaction to commercial level. As a result, a reaction scheme involving a Vilsmeier step cannot readily be scaled up.

Accordingly, improved processes providing safe, cost-effective, and efficient methods for triarylamine production are desired.

### SUMMARY

The present disclosure addresses these and other needs, by providing an improved method for the preparation of triarylamines. More particularly, this disclosure provides an improved method of producing triarylamine compounds having one or two hydroxyl groups by the reaction of an aryl amine (such as an aniline) with a halogenated aryl aldehyde. The reaction is generally a three-step process, where the halogenated aryl aldehyde is first protected to form a halogenated protected aryl aldehyde compound, followed by a second step of reacting the halogenated protected aryl aldehyde compound with an aryl amine, and removing the protecting groups, then finally in a third step the aldehyde group(s) are reduced to hydroxyl group(s).

In embodiments, the disclosure provides a process for forming a triarylamine compound, comprising:

(1) reacting a halogenated aryl aldehyde with an aldehyde protecting agent to form a halogenated protected aryl aldehyde compound

(2) reacting the halogenated protected aryl aldehyde compound with an arylamine in the presence of a suitable catalyst to form a protected aldehyde triarylamine, then removing the protecting groups with an acid, and

(3) reacting the aldehyde triarylamine with a reducing agent.

### BRIEF DESCRIPTION OF THE DRAWINGS

FIG. 1 represents a conventional process for producing a triarylamine.

FIG. 2 represents processes for producing an exemplary triarylamine according to the disclosure.

### DETAILED DESCRIPTION OF EMBODIMENTS

This disclosure is not limited to particular embodiments described herein, and some components and processes maybe varied by one of ordinary skill in the art, based on this disclosure. The terminology used herein is for the purpose of describing particular embodiments only, and is not intended to be limiting.

In this specification and the claims that follow, singular forms such as “a,” “an,” and “the” include plural forms unless the content clearly dictates otherwise. In addition, reference may be made to a number of terms that shall be defined as follows:

The term “aryl” refers, for example, to monocyclic or fused-ring polycyclic (i.e., rings which share adjacent pairs of carbon atoms) carbocyclic aromatic ring systems having about 6 to about 20 carbon atoms or more, such as phenyl, naphthyl, anthrycyl, and the like. Optionally, these groups may be substituted with one or more independently selected substituents, including alkyl, alkenyl, alkoxy, hydroxyl, nitro and further aryl groups. “Aryl” also includes heteroaryl groups, such as pyrimidine or thiophene.

The term “arylamine” refers, for example, to moieties containing both aryl and amine groups. Exemplary arylamine groups have the structure Ar—NRR′, in which Ar represents an aryl group and R and R′ are groups that may be independently selected from hydrogen and substituted and unsubstituted alkyl, alkenyl, aryl, and other suitable functional groups. The term “triarylamine” refers, for example, to arylamine compounds having the general structure NArAr′Ar″, in which Ar, Ar′ and Ar″ represent independently selected aryl groups.

“Amine” refers, for example, to an alkyl moiety in which one or more of the hydrogen atoms has been replaced by an —NH₂ group. The term “lower amine” refers, for example, to an alkyl group of about 1 to about 6 carbon atoms in which at least one, and optionally all, of the hydrogen atoms has been replaced by an —NH₂ group.

An improved three-step process for producing hydroxyl triarylamines directly from an aryl amine (such as an aniline) and a halogenated aryl aldehyde is provided. In a first step, a halogenated aryl (such as benzyl) aldehyde (such as 4-chlorobenzldehyde) is protected to form a halogenated protected aryl aldehyde compound by reacting the aldehyde with an aldehyde protecting agent (such as trimethyl orthoformate).

In a second step, the halogenated protected aryl aldehyde compound is reacted with an aryl amine (such as an aniline), such as in the presence of a suitable catalyst. For example, the catalyst may be the palladium/ligand catalyst of the Buchwald reaction. As part of the workup of this second step, the resultant protected aldehyde triarylamine is reacted with an acid to remove the protecting group. For example, the acid may be hydrochloric acid used during an acid washing as part of the workup.

Finally, in a third step the aldehyde triarylamine is reduced to form the hydroxyl triarylamine desired product, for example through reduction with sodium borohydride.

The results surrounding this process were very unexpected in that the process proceeding from the halogenated aryl aldehyde and an aryl amine to the desired hydroxyl triarylamine proceeded easily and can be scaled-up to commercial scale. This process can be used in place of the process that uses a Vilsmeier reaction. Furthermore, the process avoids not only the use of the non-commercially scaleable Vilsmeier reaction, but also avoids the use of corrosive Vilsmeier reagents such as POCl₃ or POBr₃. Therefore, this process is very practical and applicable to the preparation of hydroxyl triarylamines on an industrial scale since the present reaction produces a hydroxyl triarylamine in a short reaction time with high purity.

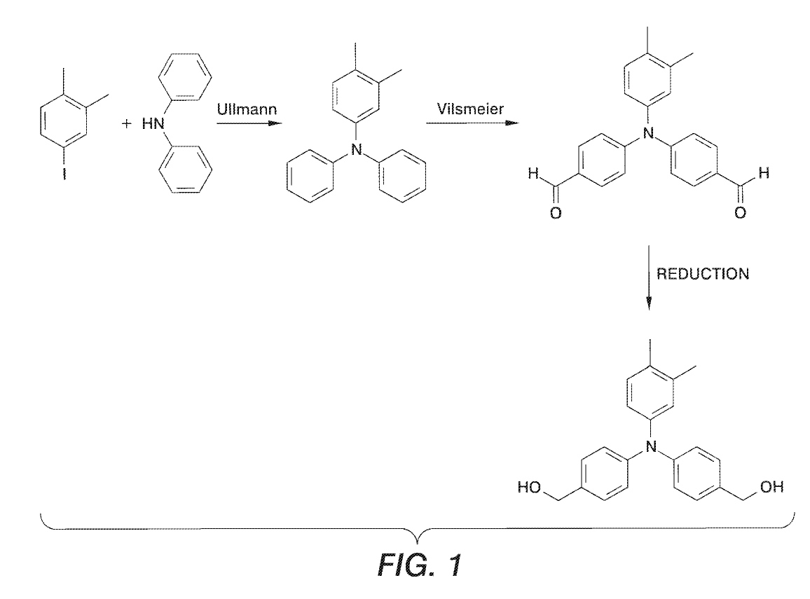

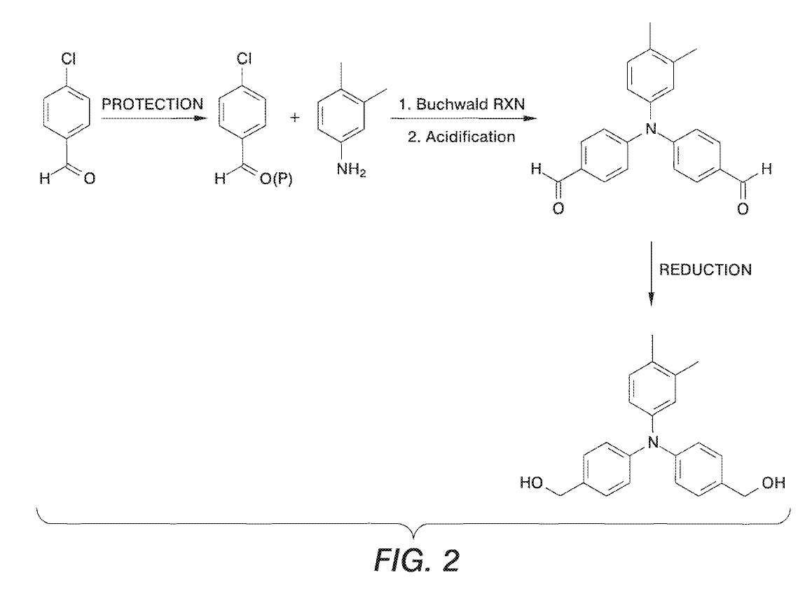

This improved process is now described in detail. Compare, for example, FIG. 1, which shows a conventional process for preparing a dihydroxyl triarylamine, with FIG. 2, which shows the present processes for preparing an exemplary dihydroxyl triarylamine.

According to the processes of the present disclosure, an aryl amine and a halogenated aryl aldehyde are used as starting materials. In general, the process of the present disclosure can be represented as:

X—Ar¹—R¹(═O)H→X—Ar¹—R¹(═OR²)H+(Ar²)ₙ(Ar³)ₘ—NH₃₋ₙ₋ₘ→→N(Ar²)ₙ(Ar³)ₘ(Ar¹—R¹(═O)H))₃₋ₙ₋ₘ→N(Ar²)ₙ(Ar³)ₘ(Ar¹—R¹—OH)₃₋ₙ₋ₘ

where X represents a halogen, such as chlorine, bromine, or iodine; n is an integer value of 1 or 2;m is an integer value of 0 or 1 provided that n+m is 1 or 2; R¹ represents an alkyl group such as from 1 to about 50 carbon atoms or from 1 to about 20 carbon atoms or from 3 to about 10 carbon atoms; R² represents a suitable aldehyde protecting group; and Ar¹, Ar² and Ar³ independently represent aryl groups.

The aldehyde protecting group R² can be any suitable aldehyde protecting group, such as an alkyl acetal or cyclic acetals such as 1,3-dioxolanes or 1,3-dioxanes or 1,3-dithianes or 1,3-dithiolanes. In particular, the aldehyde protecting group may be a group derived from trimethyl orthoformate. Any suitable aldehyde protecting group can be used, so long as it can withstand the basic conditions of the subsequent Buchwald reaction.

Ar¹, Ax² and Ar³ can be any known substituted or unsubstituted aromatic component or a substituted or unsubstituted aryl group having from 2 to about 15 conjugate bonded or fused benzene rings and could include, but is not limited to, phenyl, naphthyl, anthryl, phenanthryl, and the like. The substituents on Ar¹, Ar² or Ar³ can be suitably selected to represent hydrogen, a halogen, an alkyl group having from 1 to about 20 carbon atoms, a hydrocarbon radical having from 1 to about 20 carbon atoms, an aryl group optionally substituted by one or more alkyl groups, an alkyl group containing a heteroatom such as oxygen, nitrogen, sulfur, and the like, having from 1 to about 20 carbon atoms, a hydrocarbon radical containing a heteroatom such as oxygen, nitrogen, sulfur, and the like, having from 1 to about 20 carbon atoms, an aryl group containing a heteroatom such as oxygen, nitrogen, sulfur, and the like, optionally substituted by one or more alkyl groups, and the like.

In particular embodiments, Ar¹ is not further substituted, except by the X and R¹ groups as noted above, while Ar² is substituted by one or more groups, or visa versa. For example, in these embodiments, Ar² can be substituted by one, two, three, or more groups, such as by one or two such as two, groups, where the substituent groups can be as describe above. Even more particularly, in embodiments, Ar² is substituted by two alkyl groups, such as having from 1 to about 5 carbon atoms.

In particular embodiments, the processes of the present disclosure, including the starting materials and final product, can generally be represented as follows:

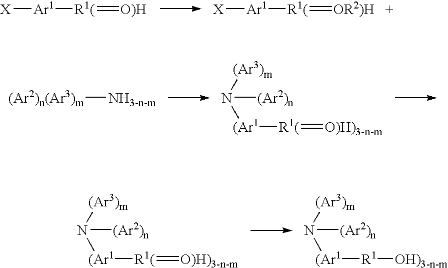

In this reaction scheme, the aryl amine and the halogenated aryl aldehyde can be any suitable compound, depending on the desired final product. Thus, for example, in the above reaction scheme, each of Ar¹, Ar² and Ar³ can be any known substituted or unsubstituted aromatic component or a substituted or unsubstituted aryl group having from 2 to about 15 conjugate bonded or fused benzene rings and could include, but is not limited to, phenyl, naphthyl, anthryl, phenanthryl, and the like. The substituents on Ar¹, Ar² and Ar³ can be suitably selected to represent hydrogen, a halogen, an alkyl group having from 1 to about 20 carbon atoms, a hydrocarbon radical having from 1 to about 20 carbon atoms, an aryl group optionally substituted by one or more alkyl groups, an alkyl group containing a heteroatom such as oxygen, nitrogen, sulfur, and the like, having from 1 to about 20 carbon atoms, a hydrocarbon radical containing a heteroatom such as oxygen, nitrogen, sulfur, and the like, having from 1 to about 20 carbon atoms, an aryl group containing a heteroatom such as oxygen, nitrogen, sulfur, and the like, optionally substituted by one or more alkyl groups, and the like.

In the first step of the process, the halogenated aryl aldehyde is converted to a halogenated protected aryl aldehyde compound to protect the aldehyde group. This reaction step generally comprises reacting the halogenated aryl aldehyde with an aldehyde protecting agent and an acid. Any suitable aldehyde protecting agents can be used, as long as the protecting group can withstand the basic conditions of the Buchwald reaction. For example, the protecting group can be any acyclic acetal such as dimethyl acetal, diethyl acetal, or dipropyl acetal and the like or the protecting group can be any cyclic acetal such as a substituted or unsubstituted 1,3-dioxolane or 1,3-dioxane or any substituted or unsubstituted 1,3-dithiane or 1,3-dithiolane.

Any suitable acid may be used in embodiments, such as p-toluenesulfonic acid, or an acidic resin such as Amberlyst-15 resin.

The reaction can be conducted in any suitable medium, such as a suitable solvent medium, as necessary or desired. In embodiments, the solvent used for the first step is desirably a polar solvent. For example, a suitable solvent for conducting the reaction is dimethylsulfoxide. Other suitable solvents include dimethylformamide, dimethylacetamide dioxane, tetrahydrofuran, acetonitrile, methanol, ethanol and the like.

In the second step of the process, the halogenated protected aryl aldehyde compound is reacted with an aryl amine to produce a protected aldehyde triarylamine compound. This reaction step generally comprises reacting the halogenated protected aryl aldehyde compound with the aryl amine under suitable reaction conditions. For example, one suitable reaction scheme is to react the materials under Buchwald reaction conditions, that is, with a base and in the presence of a suitable catalyst. The base can be any suitable base, such as an alkaline hydroxide or an alkaline alkoxide and the like. Exemplary bases that may be used in embodiments include bases having the general formula MOR, in which O is oxygen, M is a metal atom, and R is a hydrogen or an alkyl group. M is a metal selected from potassium, sodium, lithium, calcium, magnesium and the like; and R is a hydrogen or a straight or branched alkyl group selected from methyl, ethyl, propyl, butyl, pentyl, hexyl, heptyl, octyl, nonyl, and decyl groups, such as methyl, ethyl, n-propyl, isopropyl, n-butyl, isobutyl, tert-butyl, octyl, decyl and the like. Suitable bases include potassium hydroxide, potassium tert-butoxide salt, sodium tert-butoxide, and sodium tert-pentoxide. The catalyst is also not particularly limited, and suitable catalysts include those that are known or discovered to be useful for formation of nitrogen-carbon bonds. For example, suitable catalysts include ligated palladium catalysts, such as those disclosed by Buchwald et al. and Hartwig et al. (see, e.g., J. Org. Chem. 2000, 65, 5327-5333, the entire disclosure of which is incorporated herein by reference).

In an embodiment of the present disclosure, an example of a suitable catalyst is palladium acetate ligated with tri-t-butylphosphine in the presence of a base. One specific suitable catalyst is 2,4,6-trioxa-1,3,5,7-tetramethyl-8-phosphaadamantane, which is manufactured as Cytop-216 (Cytec Industries). However, it will be apparent to those skilled in the art that other ligands, such as any tertiary phosphine ligand such as biaryldialkylphosphine or trialkyl phosphine ligands, or N-heterocyclic carbene complexes could also be used to produce suitable results (from the point of view of conversion and yield), and thus would be suitable to ligate palladium or other metals and thus act as catalysts for the process described in this disclosure.

Alternatively, another suitable reaction scheme for the second step is to react the materials under Ullmann reaction conditions, that is, in the presence of a suitable catalyst and a base, optionally in the presence of a high boiling hydrocarbon such as decane as a solvent. Examples of the suitable base that can be used in the process include alkali metal hydroxides such as lithium hydroxide, sodium hydroxide, potassium hydroxide, rubidium hydroxide, and cesium hydroxide; alkali metal carbonates such as lithium carbonate, sodium carbonate, potassium carbonate, rubidium carbonate, and cesium carbonate: alkali metal phosphates such as trilithium phosphate, trisodium phosphate, and tripotassium phosphate; and alkali metal alkoxides such as sodium methoxide, sodium ethoxide, potassium methoxide, potassium ethoxide, lithium tert-butoxide, sodium tert-butoxide, and potassium tert-butoxide. Of these bases, the alkali metal alkoxides may be added as they are to the reaction system or may be prepared from the alkali metals, alkali metal hydrides, alkali metal hydroxides, or the like and an alcohol and used.

Specific examples of suitable bases are sodium hydroxide, potassium hydroxide, sodium carbonate, and potassium carbonate. These bases can generally be used in an amount of about 1 to about 4 equivalents, such as about 3.2 to about 2.0 equivalents, to the aromatic amine.

Examples of suitable catalysts include copper catalysts, such as copper powder, copper (I) chloride, copper (II) chloride, copper (I) bromide, copper (II) bromide, copper iodide, copper (I) oxide, copper (II) oxide, copper sulfate, copper nitrate, copper carbonate, and copper (II) hydroxide. Specific suitable examples include copper(I)oxide, copper chlorides, copper bromides, and copper iodide. The amount of these copper catalysts to be used is generally about 0.003 to about 0.3 mol, such as about 0.03 to about 0.2 mol, per mol of the aromatic halogen compound. If desired or necessary, a promoter such as lithium iodide, sodium iodide, potassium iodide, rubidium iodide, cesium iodide, or the like may be added. In the case where these promoters are added, they can be used in an amount of about 0.001 to about 0.5 mol, such as about 0.01 to about 0.2 mol, per mol of the aromatic halogen compound.

As a modification of the Ullmann reaction, a ligand-accelerated Ullmann reaction can also be used. This reaction is generally also conducted in the presence of a catalyst, such as the catalysts described above for the standard Ullmann reaction, but also in the presence of a bidentate ligand of the following formulas:

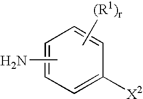

wherein 0 or 1 of the carbon atoms are replaced with N, or

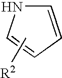

wherein from 0 to 3 of the carbon atoms are replaced with N, or the compound is benzo-fused and 0 to 2 of the carbon atoms of the five-membered ring are replaced with N; and wherein:
X¹ is selected from Cl, Br, I, and SCN;
X² is selected from Br or I;
R¹ is selected from H, Cl, F, Br, I, C₁₋₄ alkyl, C₁₋₄ alkoxy, C₁₋₄ alkylene-O—C₁₋₄ alkyl, NH₂, NH(C₁₋₄ alkyl), N(C₁₋₄ alkyl)₂, C₁₋₄ alkylene-NH₂, C₁₋₄ alkylene-NH(C₁₋₄ alkyl). C₁₋₄ alkylene-N(C₁₋₄ alkyl)₂, C₃₋₁₀ carbocycle substituted with 0-2 R³, 5-6 membered heterocycle comprising carbon atoms and 1-4 heteroatoms selected from N, O, and S and substituted with 0-2 R³;
R² is selected from H, Cl, F, Br, I, C₁₋₄ alkyl. C₁₋₄ alkoxy, C₁₋₄ alkylene-O—C₁₋₄ alkyl, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, C₃₋₁₀ carbocycle substituted with 0-2 R³, 5-6 membered heterocycle comprising carbon atoms and 1-4 heteroatoms selected from N, O, and S and substituted with 0-2 R³;
R³ is selected from Cl, F, Br, I, C₁₋₄ alkyl, C₁₋₄ alkoxy, C₁₋₄ alkylene-O—C₁₋₄ alkyl, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
r is 1 or 2; and,
the bidentate ligand is a hydrolytically stabile ligand that ligates with Cu(I) and comprises two heteroatoms selected from N and O.

In embodiments, suitable specific examples of the bidentate ligand include tetramethylethylenediamine (TMED), 2,2′-dipyridyl (DPD), 8-hydroxyquinoline (HQL), 1,10-phenanthroline (PNT), 8-hydroxyquinoline (HQL), and 1,10-phenanthroline (PNT). The bidentate ligand can be used in any desired and suitable amount, such as from about 0.001 to about 0.5 equivalents, based on the molar amount of aniline present.

The reaction of the second step of the process can be carried out in the presence of the catalyst, and can be conducted in continuous mode. However, the reaction may be conducted in batch mode. For example, the reaction can be carried out for a period of from about 2 to about 30 hours or more, such as a reaction time of from about 10 to about 14 hours.

The reaction of the second step of the process can be carried out in a suitable solvent, such as toluene, xylene, decane, other hydrocarbon solvents (either aromatic or saturated hydrocarbons), or mixtures thereof. The choice of solvent can be decided based on the solubility of the starting materials, intermediates, and final products, and will be readily apparent or within routine experimentation to those skilled in the art. Furthermore the choice of solvent can be decided based on the desired operating temperature range. The described process is exothermic and precautions should be taken to ensure that the solvent chosen is capable of dispersing the produced heat by, for example, refluxing and cooling at such a rate so as to control the exotherm. The reaction should be conducted under an atmosphere of inert gas (such as nitrogen or argon) so as to preclude deactivation of catalyst or base by oxygen or atmospheric moisture.

As part of the workup of the second step, the protected aldehyde triarylamine compound is reacted with an acid to remove the protecting group, and thereby forms a aldehyde triarylamine compound. Any suitable acid may be used, such as hydrochloric acid, that creates a pH of from about 6.9 to about 1. Other acids that may be used could include acetic acid, or sulfuric acid

In the third step of the process, the aldehyde triarylamine is reacted with a reducing agent to form the hydroxyl triarylamine. Suitable reducing agents include known metallic hydrides, specifically borohydrides for example sodium triacetoxyborohyride, sodium cyanoborohydride or sodium borohydride. This step can take place in a standard nonpolar hydrocarbon solvent, such as ethanol.

After the reaction is completed, suitable separation, filtration, and/or purification processes can be conducted, as desired to a desired purity level. For example, the desired hydroxyl triarylamine product can be subjected to conventional organic washing steps, can be separated, can be decolorized (if necessary), treated with known absorbents (such as silica, alumina, and clays, if necessary) and the like. The final product can be isolated, for example, by a suitable recrystallization procedure. The final product can also be dried, for example, by air drying, vacuum drying, or the like. All of these procedures are conventional and will be apparent to those skilled in the art.

The hydroxyl triarylamine produced by this process can be further processed and/or reacted to provide other compounds for their separate use. For example, the hydroxyl triarylamine can be further processed and/or reacted to provide charge-transport materials or other compounds useful in electrostatographic imaging members.

### EXAMPLES

The disclosure will be illustrated in greater detail with reference to the following Example, but the disclosure should not be construed as being limited thereto. In the following example, all the “parts” are given by weight unless otherwise indicated.

### Example

Protection of 4-chlorobenzaldehyde: 4-chlorobenzaldehyde, in the amount of 100 grams, was placed into a 500 mL round bottom flask and dissolved in 389 mL of trimethyl orthoformate. Amberlyst-15 ion-exchange resin was then added and the mixture was stirred under argon for a period of approximately 12 hours at roughly 22° C. Thereafter, the mixture was filtered to remove the Amberlyst-15 resin, and the solvent was removed under reduced pressure. The remaining reaction product was then purified by distillation at 86° C. under reduced pressure to produce a while oil. The reaction yielded 127 grams of white oil, for a 96% yield.

### Buchwald Reaction:

To a 500 mL 3-necked round bottom flask equipped with a mechanical stirrer, argon inlet, thermometer and reflux condenser was placed 0.55 grams of palladium acetate and 0.52 grams of 2,4,6-trioxa-1,3,5,7-tetramethyl-8-phosphaadamantane (Cytop 216), which were dissolved in 25 mL of xylene under argon. To this was added 14.76 grams of 3,4-dimethylaniline, 50 grams of the protected 4-chlorobenzaldehyde and 35.11 grams of sodium, tert-butoxide. The mixture was stirred under argon at 120° C. for 12 hours.

The mixture was then cooled and washed twice with a 20% HCl solution. The organic layer was then collected, dried with MgSO₄ and then treated with 20 grams of an acidified clay and 20 grams of an alumnia at 100° C. for 30 minutes. Afterwards, the solids were filtered and the filtrate was concentrated under reduced pressure to produce a yellow solid in 86% yield.

### Reduction

To a 500 mL round bottom flask was placed 11 grams of the triphenylamine from the previous step, which was dissolved in 50 mL of ethanol under argon. One tenth of a gram of sodium hydroxide was then added and allowed to dissolve, after which 1.4 grams of sodium borohydride was added. The mixture was stirred at roughly 22° C. for approximately 2 hours. The mixture was then poured into 400 mL of water in a standard beaker, and this mixture was stirred for roughly 12 hours. A yellow solid precipitated out, which was collected by filtration and washed with 200 mL of water. The resultant dihydroxymethylated triphenylamine was recrystalized twice from toluene and dried to produce a white powder in 82% yield.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

What is claimed is:

1. A process for forming a hydroxyl triarylamine compound, comprising
(1) reacting a halogenated aryl aldehyde with an aldehyde protecting agent to form a halogenated protected aryl aldehyde compound;
(2) reacting the halogenated protected aryl aldehyde compound with an arylamine in the presence of a catalyst and a suitable base to form a protected aldehyde triarylamine, then removing a protecting group with an acid to form an aldehyde triarylamine; and
(3) reacting the aldehyde triarylamine with a reducing agent.

2. The process of claim 1, wherein the halogenated aryl aldehyde and the arylamine are represented as follows:
X—Ar¹—R¹(═O)H→X—Ar¹—R¹(═OR²)H+(Ar²)ₙ(Ar³)ₘ—NH₃₋ₙ₋ₘ→N(Ar²)ₙ(Ar³)ₘ(Ar¹—R¹(═O)H))₃₋ₙ₋ₘ→N(Ar²)ₙ(Ar³)ₘ(Ar¹—R¹—OH)₃₋ₙ₋ₘ
wherein:
X is a halogen,
n is an integer value of 1 or 2,
m is an integer value of 0 or 1, provided that n+m is 1 or 2,
R¹ represent an alkyl group of from 1 to 50 carbon atoms,
R² represents a suitable aldehyde protecting group, and
Ar¹, Ar² and Ar³, which can be the same or different, are selected from the group consisting of tri-, di- or mono-substituted or unsubstituted aromatic components, and substituted or unsubstituted aryl groups having from 2 to about 15 conjugate bonded or fused benzene rings, wherein a substituent on the aryl groups Ar¹, Ar² and Ar³ is one or more of the group consisting of hydrogen, a halogen, an alkyl group having from 1 to about 20 carbon atoms, a hydrocarbon radical having from 1 to about 20 carbon atoms, an aryl group, an aryl group substituted by one or more alkyl groups, an alkyl group containing a heteroatom and having from 1 to about 20 carbon atoms, a hydrocarbon radical containing a heteroatom and having from 1 to about 20 carbon atoms, an aryl group containing a heteroatom, and an aryl group containing a heteroatom substituted by one or more alkyl groups.

3. The process of claim 1 wherein the step (2) is a Buchwald reaction.

4. The process of claim 1, wherein the catalyst is a palladium catalyst.

5. The process of claim 1, wherein the catalyst is a palladium ligated catalyst.

6. The process of claim 5, wherein the palladium ligated catalyst is 2,4,6-trioxa-1,3,5,7-tetramethyl-8-phosphaadamantane.

7. The process of claim 1 wherein the step (2) is an Ullmann reaction,

8. The process of claim 1, wherein the catalyst is a copper catalyst,

9. The process of claim 1, wherein the catalyst is a copper catalyst ligated with a bidentate ligand.

10. The process of claim 9, wherein the the bidentate ligand is selected from the group consisting of: tetramethylethylenediamine (TMED), 2,2′-dipyridyl (DPD), 8-hydroxyquinoline (HQL), 1,10-phenanthroline (PNT), 8-hydroxyquinoline (HQL), and 1,10-phenanthroline (PNT).

11. The process of claim 1, wherein the step (2) takes place at a temperature from about 100° C. to about 150° C.

12. The process of claim 1 wherein the aldehyde protecting agent is dimethyl acetal.

13. The process of claim 1 wherein the reducing agent is selected from the group consisting of sodium triacetoxyborohyride, sodium cyanoborohydride and sodium borohydride.

14. The process of claim 1, wherein the step (2) is carried out in a non-polar solvent.

15. The process of claim 2, wherein Ar¹, Ar² and Ar³ are phenyl groups.

16. The process of claim 14, wherein Ar² is a di-substituted phenyl group.

17. The process of claim 2, wherein X is chlorine.

18. The process of claim 1, wherein the step (1) is conducted in the presence of an ion-exchange resin.

19. The process of claim 1, further comprising recrystallizing the hydroxyl triarylamine from toluene, and then drying the hydroxyl triarylamine.

20. A process for forming a dihydroxymethylated triphenylamine compound, comprising
(1) reacting a 4-chlorobenzaldehyde with trimethyl orthoformate to form a protected 4-chlorobenzaldehyde compound,
(2) reacting the protected 4-chlorobenzaldehyde compound with 3,4-dimethylaniline in the presence of a Buchwald palladium/ligand catalyst and a base, and then an acid, and
(3) reacting the resultant compound with a reducing agent.
